(19) United States
(12) Patent Application Publication
(10) Pub. No.: US 2025/0213776 A1
(43) Pub. Date: Jul. 3, 2025

(54) PORTABLE BASE FOR A BOWEL IRRIGATION SYSTEM

(71) Applicant: Coloplast A/S, Humlebaek (DK)

(72) Inventors: Stephanie Knoedler, Nivaa (DK); Kristoffer Zeuthen, Copenhagen OE (DK); Nina Hoegh Jensen, Copenhagen N (DK)

(21) Appl. No.: 18/853,442

(22) PCT Filed: Mar. 29, 2023
(86) PCT No.: PCT/DK2023/050066

§ 371 Date: Oct. 2, 2024

(30) Foreign Application Priority Data
Apr. 8, 2022 (DK) PA202270194

(51) Int. Cl. A61M 3/02

(52) U.S. Cl. CPC A61M 3/0245

(57) ABSTRACT

A portable base for a system for irrigation of the bowels of a user is provided. The portable base (20) for a system (10) for irrigating the bowels of a user, where the portable base comprises a canister unit (22) and a lid unit (24), the canister unit comprises a bottom portion (25), a side wall (30) and a neck portion (38) for forming an inner volume (78) for containing an irrigation liquid (55), wherein the neck portion circumscribes an annular opening (40) around a central vertical axis (C) of the portable base, the lid unit (24) is adapted for closing of the annular opening (40) of the canister unit, the side wall (30) comprises a at least partly see-through portion (57) and wherein the lid unit comprises one or more lights (53) being adapted to light up the inner volume (78) of the canister unit (22).

A portable base for a system for irrigation of the bowels of a user as well as a system of bowel irrigation is provided. The portable base comprises a canister unit for an irrigation liquid and a lid unit for engaging with the canister unit.

### BACKGROUND

Control of voluntary bowel functions is frequently limited or absent in patients suffering from certain disabilities, such as spinal injuries, multiple sclerosis, or spina bifida. Such lack of control of voluntary bowel functions typically results in faecal incontinence or intractable constipation, as patients have significantly reduced ability to sense the presence of faeces in the colon terminal part and the rectum and to sense the evacuation stimulus.

It is known to accomplish bowel emptying by irrigation (i.e. flushing) of the bowels via the rectum, by an irrigating fluid, such as tap water or saline, which is provided through an intermittent catheter with a tip which is configured and sized for insertion into the rectum (the rectum being the terminal section of the alimentary canal; from the sigmoid flexure to the anus), where it remains in a fixed position and is held by an inflatable element, which may be inflatable by air or by water with a pump. Once the rectum or stoma has been flushed with the irrigating liquid, the inflatable element is removed, and the liquid and faeces can evacuate. Some known irrigation systems suffer from being bulky, complicated and difficult to handle and difficult to clean properly after use. Moreover, some users of irrigation systems have limited vision due to medical conditions and therefore may have difficulty in viewing components of the irrigation system, including particularly in the process of filling and/or emptying irrigation liquid from the container and assessing a remaining volume of irrigation liquid and when the container is empty.

Thus, users of irrigation systems and health care professionals alike would welcome improvements to components of bowel irrigation systems.

### BRIEF DESCRIPTION OF THE DRAWINGS

The accompanying drawings are included to provide a further understanding of embodiments and are incorporated in and constitute a part of this specification. The figures illustrate embodiments and together with the description explain principles of embodiments. Other embodiments and many of the intended advantages of all embodiments will be readily appreciated as they become better understood by reference to the following detailed description.

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment does not necessarily have all the effects or advantages shown. Conversely, an effect or an advantage described in conjunction with a particular embodiment is not necessarily limited to that embodiment and may be practiced in other embodiments, even if not so illustrated, or if not so explicitly described.

The figures include the following:

FIG. 1 illustrates a perspective view of a system for irrigating the bowels of a user including a portable base.

FIG. 2 illustrates a schematic side view of a portable base.

FIG. 3 illustrates a schematic side view of a portable base.

FIG. 4 illustrates a schematic side view of a portable base.

FIG. 5 illustrates a schematic side view of a lid unit and a canister unit.

FIG. 6 illustrates a schematic side and bottom view of a simplified version of a lid unit comprises a light.

FIG. 7 illustrates a schematic side and bottom view of a simplified version of a lid unit comprising four lights distributed in an annular pattern,

FIG. 8 illustrates a schematic side and a perspective view of a canister unit comprising an inclined protrusion.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” and equivalent, is used with reference to the orientation of the Figure(s) being described. Because components of embodiments can be positioned in a number of different orientations, the directional terminology is used for purposes of illustration and is not necessarily limiting. It is to be understood that other embodiments may be utilized, and structural or logical changes may be made without departing from the scope of the present invention. The following detailed description, therefore, is not to be taken in a limiting sense, and the scope of the present invention is defined by the appended claims.

It is to be understood that individual features of the various exemplary embodiments described herein may be combined with each other, unless specifically noted otherwise.

In this disclosure, solid and liquid wastes from the bowels of a person may be referred to as both “output,” “waste(s),” and “fluids” interchangeably. A person using the irrigation system may be referred to as “patient” or “user”. However, in some contexts, “user” can also relate or refer to a health care professional (HCP), such as a doctor/physician, nurse, or other person.

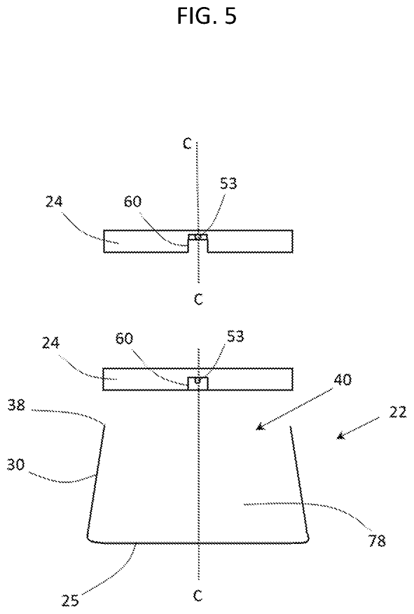

In this disclosure, ‘axial direction’ should generally be considered as vertical seen in relation to a top and bottom of the portable base. A central vertical axis is illustrated in FIG. 5, by a dotted line marked C. This central vertical axis corresponds to the axial direction. The radial direction should be understood as transverse to the axial direction. In some sentences, the words “inner” and “outer” can be used. These qualifiers should generally be perceived with reference to the radial direction, such that a reference to an “outer” element means that the element is farther away from a centre portion of a component than an element referenced as “inner”.

The use of phrases such as (but not limited to) “substantially” and “approximately” as qualifiers of certain features or effects in this disclosure, is intended to mean that any deviations are within tolerances that would normally be expected by the skilled person in the relevant field.

The use of the word “generally” as a qualifier to certain features or effects in this disclosure is intended to mean—for a structural feature: that a majority or major portion of such feature exhibits the characteristic in question, and—for a functional feature or an effect: that most outcomes involving the characteristic provide the effect, but that exceptionally some outcomes may not provide the effect.

The phrase “adjacent” should be interpreted as being “close to and/or in contact with, or temporarily brought into contact with, but not attached to [something].”

In the present disclosure, “portable” is to be understood as being easy to carry or move, so that a “portable” element can be used in different places and can be easily and conveniently transported. Particularly, this should be understood in relation to some types of available irrigation systems for the bowels of a user including components which are large and bulky and intended for bedridden and/or hospitalized users only.

The irrigation liquid for an irrigation system can be temperate tap water or in some cases a saline physiological solution.

During preparation and during irrigation it may be necessary for a user to assess the amount of irrigation liquid in a canister unit. This may for some users be quite challenging due to several factors like lack of space around the portable base and the canister unit, the position of the canister unit in relation to the user, as the canister unit may be positioned on the floor next to the user sitting e.g., on a toilet, and therefore the canister unit may be positioned significant below the eye level which normally is a preferred position for assessing a level of material in a container. Also, the amount of ambient light level and/or light conditions may cause some challenges for some users. In a first aspect, the present disclosure relates to a portable base for a system for irrigating the bowels of a user,

the portable base comprises a canister unit and a lid unit, where the canister unit comprises a bottom portion, a side wall and a neck portion for forming an inner volume for containing an irrigation liquid, wherein the neck portion circumscribes an annular opening around a central vertical axis of the portable base, the lid unit is adapted for closing of the annular opening of the canister unit, the side wall comprises an at least partly see-through portion and wherein the lid unit comprises one or more lights being adapted to light up the inner volume of the canister unit.

Thereby, the emitted light from the light(s) in the lid unit will brighten up the inner volume and the light will also enhance the transition between the inner volume being filled with air and the inner volume containing irrigation liquid due to refraction of the light in the different materials in the canister unit. Hereby, the volume of irrigation liquid in the canister unit may for some users be easier to assess, and for some users it may be easier to determine when the canister unit is empty for irrigation liquid. The light is shining on the surface of the irrigation liquid in the container, which will increase the visibility of the level of liquid in the container, even from a position above the portable canister unit, e.g., when the container is standing on the floor next to a toilet. Thereby a user may quicker and/or easier assess whether there is any liquid within the inner volume and the volume of said liquid.

The canister unit comprises the inner volume for the irrigation liquid. The canister unit includes a neck portion circumscribing an annular opening around a central vertical axis of the portable base. In embodiments, the neck portion comprises a vertical flange extending generally parallel to a central vertical axis of the portable base. In embodiments, an upper part (area or zone) of the flange includes a tapering, i.e., it is at an angle to the central vertical axis. In embodiments, the neck portion transitions into a shoulder portion of the canister unit. The transition between the neck portion and the shoulder portion is located at a lower part (area or zone) of the neck portion. In embodiments, the shoulder portion of the canister unit (further or in turn) transitions into a sidewall of the canister unit. The transition between the shoulder portion and the sidewall of the canister unit is located at a lower part (area or zone) of the shoulder portion and can further be located radially outermost on the shoulder portion. In embodiments, the neck portion is offset radially inwardly from the sidewall of the canister unit by (or at) the shoulder portion and the neck portion is radially closer to the central vertical axis than is the sidewall of the canister unit. In embodiments, the neck portion includes one or more threads or thread segments in the form of protrusions and/or ledges extending generally radially from and perpendicular to the vertical flange.

In embodiments, the side wall comprises a radially extending inclined protrusion comprising a protrusion face, and the at least partly see-through portion is arranged on the protrusion face. The protrusion may function as a handle, when rotating the lid for opening and closing the portable unit.

In other words, the protrusion forms a sloped wall portion extending radially outwards in a gradually increasing inclination from the top of the canister towards the bottom of the canister.

In embodiments, the canister unit comprises a volumetric scale arranged at the at least partly see-through portion of the side wall.

In embodiments, the canister unit is made of see-trough material.

In embodiments, the protrusion face of the inclined protrusion in axial direction is angled 2-45 degrees relative to the central vertical axis, such as 5-40, 6-35, 7-30, 8-25, 9-20, 10-15 degrees.

In embodiments, the protrusion face of the inclined protrusion is angled such that the radial distance to the central vertical axis C is increasing towards to the bottom portion of the canister unit. The canister unit is adapted to hold a volume of irrigation liquid inside the canister in the inner volume, and by the inclined protrusion face a user may be able to determine whether there is any liquid in the canister unit, even from a position above the portable canister unit, e.g., when the container is standing on the floor next to a toilet.

In embodiments, the canister unit is adapted to stand by itself on the bottom of the canister unit on a generally level surface of the surroundings. This is useful for making the portable base in its entirety stable when standing on a surface during use.

In embodiments, the sidewall of the canister unit extends substantially parallel to the central vertical axis. Thus, the canister unit generally takes the shape of a cylinder when viewed in cross-section.

In embodiments, the annular opening of the canister unit has a diameter of between 100-150 mm. In one embodiment, the annular opening of the canister unit has a diameter of 120 mm. These embodiments are particularly suitable for providing a possibility of thoroughly cleaning the insides of the canister unit, e.g., by allowing a user to insert a hand entirely through the annular opening or alternatively a brush or other cleaning tool.

The neck portion can be understood as delimiting, or defining or surrounding, the extent (or size) of the annular opening of the canister unit. Particularly, the annular opening provides a passage which is highly useful when cleaning or disinfecting the canister unit. In embodiments, the annular opening is relatively large, which greatly assists a user in cleaning the inner volume of the canister unit by inserting a hand or the fingers thereinto for cleaning. The annular opening can also at least partly provide a passage for filling and emptying irrigation liquid into and from the canister unit. The portable base includes a lid unit configured for closing of the annular opening of the canister unit. The lid unit has an axial extent in a direction along the central vertical axis between a top and a bottom of the lid unit. Thus, the lid unit has a height in a direction parallel to the central vertical axis. The lid unit is configured for attachment to (or engagement with) the canister unit over the annular opening (to close the canister unit).

The canister unit comprises a volumetric scale arranged at the at least partly see-through portion of the side wall.

In embodiments, the lid unit includes at least one internal chamber. The at least one internal chamber is adapted to contain one or more control elements of a system for irrigating the bowels of a user. In embodiments, one or more control elements are held in the internal chamber and/or partially on an outer surface of the lid unit. In embodiments, a top of the lid unit comprises one or more connections and/or interfaces (e.g., spigots or studs) for engagement and correspondence with other elements and/or components of a system for irrigating the bowels of a user. In embodiments, the lid unit includes a passage for allowing irrigation liquid from the canister unit to exit from the canister, typically by means of a pumping action provided by a pump of the system for irrigating the bowels of a user. In one embodiment, the lid unit includes two separate, internal chambers. In embodiments, the lid unit includes more than two separate, internal chambers. In embodiments, one internal chamber of the lid unit includes or houses a pump and forms part of a system for irrigating the bowels of a user.

In embodiments, the canister unit is made of see-trough material. Parts of the canister unit may be made of partially transparent material, thus still allowing a user to assess a level of irrigation liquid.

In embodiments, the canister unit comprises a frustoconical shape. Generally, the bottom portion is wider than the neck portion. The advantage of this shape being a more stable portable base and canister unit, and the content within the inner volume of the canister unit is visually easier for a user to assess, thus also reading the volumetric scale reflecting a volume of any content in the canister unit.

In embodiments, at least ⅔ of the side wall of the canister unit in axial direction is angled 2-45 degrees relative to the central vertical axis, such as 5-40, 6-35, 7-30, 8-25, 9-20, 10-15 degrees.

The side wall may comprise an extent in axial direction of 60-90 mm, such as 70 mm.

Hereby, the portable base is more stable, moreover the assessment of any content in the canister unit is more optimal for the user. However, the portable base may require less storage place when the side wall has a more vertical extent and a smaller degree of tapering. A canister unit having 10-15 degrees angled sidewall has several of the advantages mentioned above. One advantageous effect of the provision of the angled sidewall of 10-15 degrees is that the inner volume and any content may be visible for a user viewing the portable base and the canister unit from above. The angled container helps for better visibility.

In embodiments, the neck portion is radially closer to the central vertical axis than the sidewall of the canister unit.

In embodiments, the one or more lights are built-in LED Lights.

Among other advantages, the lid unit can additionally house one or more delicate control components in one or more internal chambers, e.g., a CPU and/or other electronics, whereby it is useful to avoid movements of the entire lid unit to reduce unnecessary shifting and stress on the housed components from the externally incurred rotation forces.

In embodiments, one light is positioned in the lid unit within a radial distance of 30 mm or less from the central vertical axis.

In embodiments, the one or more lights are arranged evenly in an annular ring-shaped pattern within a radial distance of 10-60 mm from the central vertical axis.

In embodiments, the one or more lights emit coloured light, such as red, blue, green and/or yellow light.

In embodiments, the one or more lights emit flashing light.

Generally, the one or more lights in the lid unit may emit coloured light, such as red, blue, green, violet, orange, yellow and/or white light. The light may be used for alarming or signalling to the user, indicating one or more functions and/or instructions to a user of the system, such as different states within the irrigation process, e.g., the possibility of releasing a flow of irrigation liquid from the canister unit 22 for commencement of a bowel irrigation cycle. The light may be used as a warning signal. E.g. slow pulsation, in case of a specific water-pressure.
Generally, the one or more lights may emit flashing light. The flashing light may comprise slow, fast or with varying frequencies. The flashing may be progressive in intensity.

In embodiments, the lid unit comprises an on/off button for turning the one or more lights off and on.

### Detailed Description of the Drawings

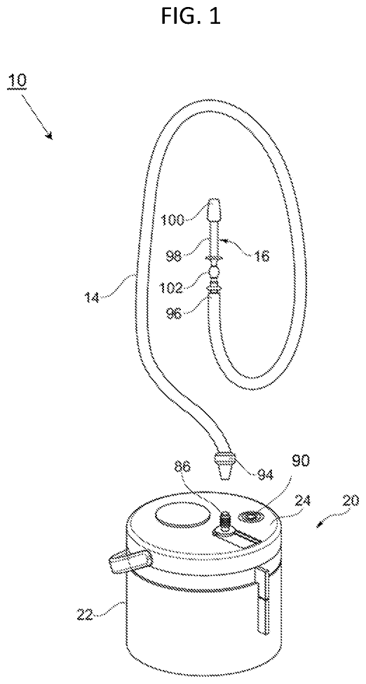

FIG. 1 is a perspective view illustrating a system 10 for irrigating the bowels of a user including a portable base 20 comprising a canister unit 22 and a lid unit 24. Furthermore, the system comprises tubing 14 and a catheter or anal probe 16. A first end portion 94 of the tubing 14 is configured for connection and/or attachment with the connection 86 on the lid unit 24. A second end portion 96 of the tubing 14 is configured for connection and/or attachment with the anal probe 16. These connections assist in providing fluid communication between the irrigation liquid in the canister unit 22 and the anal probe 16 via the tubing 14. The catheter or anal probe 16 is configured to be inserted into the rectum of a user. The anal probe 16 includes a catheter portion 98 for insertion into the rectum of the user to release irrigation liquid via the catheter into the bowels of the user. The catheter portion 98 includes a catheter tip 100. The anal probe 16 further includes a handle portion 102, which is connectable to the tubing 14. The lid unit 24 comprises an on/off button 90 for manually turning the one or more lights on and off, thus emitting light into the inner volume of the canister unit 22.

Although not illustrated in FIG. 1, the system 10 can optionally include a set of instructions for use, and/or a container for storing the components of the system.

The portable base 20 comprises a canister unit 22, which is self-supporting and a lid unit 24 for closing of an annular opening (illustrated in FIG. 5). In the perspective view of FIG. 1, the portable base 20 is shown in a sealed (or closed) configuration with the lid unit 24 placed “on top of” and closing the canister unit 22. The canister unit 22 is adapted to hold a volume of irrigation liquid inside the canister in the inner volume 55. It is to be understood that the lid unit 24 is removably connected to the canister unit 22 and thereby, by removing the lid unit 24, access to the annular opening is given. In the embodiment shown in FIG. 1, the lid unit 22 includes a handle attached to the lid unit which can be used when lifting off the lid unit 24 from the canister unit 22 and for moving of the portable base 20 around when it is in the sealed configuration.

The lid unit 24 can be configured to hold one or more control elements or control components of a bowel irrigation system.

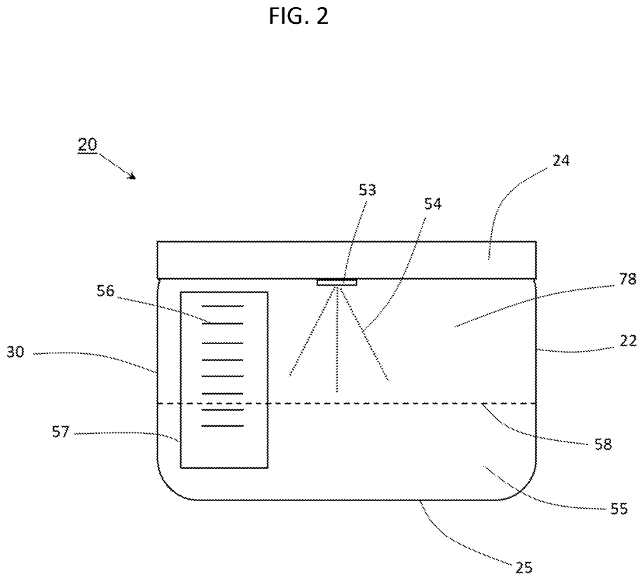

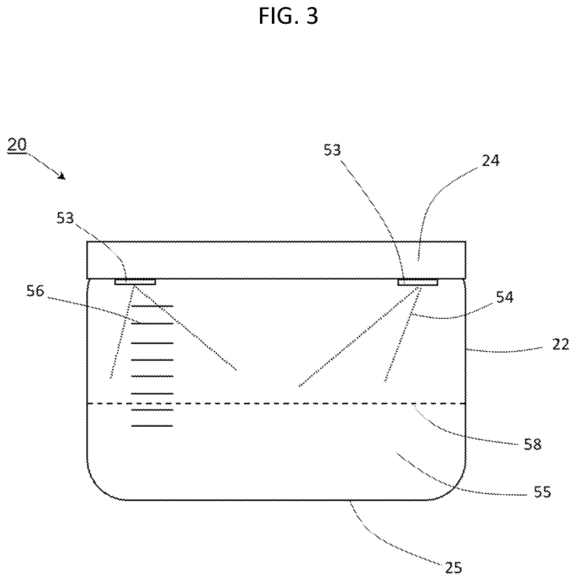

FIGS. 2 and 3 schematically illustrate a schematic side view of a portable base 20 and where the lid unit 24 comprises one or more lights. FIG. 2 illustrates a portable base 20 comprising a canister unit 22 and a lid unit 24. The canister unit 22 having a bottom portion 25 and a side wall 30 forming an inner volume 78 for containing irrigation liquid 55. The content of the inner volume is visible for a user due to a see-through portion 57 having a volumetric scale 56. The level of the irrigation liquid 55 is illustrated by dotted line 58. The volumetric scale 56 is marked on the canister unit 22 by horizontal lines to represent units of measurement and used to measure the volume of irrigation liquid 55.

The lid unit 24 comprises a light 53, such as LED light, which illuminate the inner volume 78 of the canister unit and any content therein. The emit of light is illustrated by dotted lines 54, when the portable base is in closed configuration having the lid unit 24 mounted on the canister unit 22.

The canister unit 22 is adapted to hold a volume of irrigation liquid 55 inside the canister unit in the inner volume 78. The level of irrigation liquid is illustrated by dotted line 58. The canister unit 22 comprises a volumetric scale 56 arranged at a see-through portion 57 of the side wall 30. The see-through portion 57 may be partially transparent.

FIG. 3 illustrates a similar side view of the portable base 20 as illustrated in FIG. 2. The canister unit 22 is made of see-through material. A user may thus inspect the inner volume through the side wall of the canister unit. The lid unit 24 comprises two lights 53 illuminating the inner volume of the canister unit 22 and the irrigation liquid 55, thus the visibility of the level 58 of the irrigation liquid 55 in the canister unit 22 is enhanced.

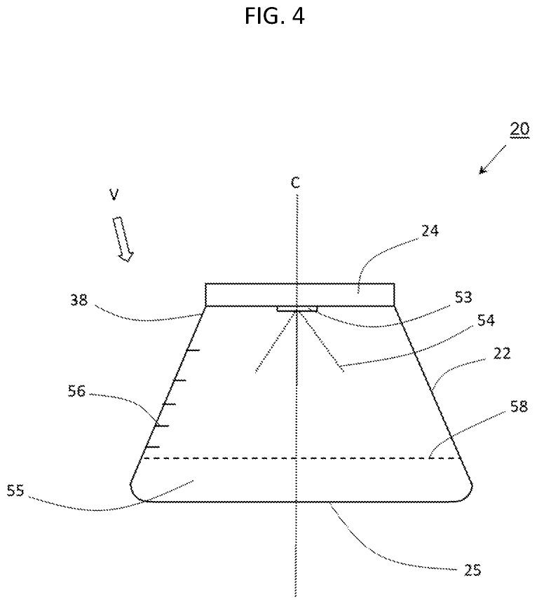

FIG. 4 illustrates a schematic side view of a portable base 20. The portable base 20 comprises a frustoconical shaped canister unit 22, where the bottom portion 25 is wider than the neck portion 38 of the canister unit. An arrow marked V illustrates the direction from where a user may view the portable base 20, e.g. when the portable base is positioned on the floor next to a user sitting on a toilet.

The sidewall of the canister unit 22 in axial direction is angled approximately 10-15 degrees relative to the central vertical axis C.

FIG. 5 illustrates a schematic side view of a lid unit 24 and a canister unit 22. The canister unit 22 comprises an annular opening 40 and an inner volume 78. The illustrated neck portion 38 is radially closer to the central vertical axis C than the sidewall 30 of the canister unit 22. The light 53 is arranged in a light housing 60 positioned centrally in the lid unit 24 along the central vertical axis C.

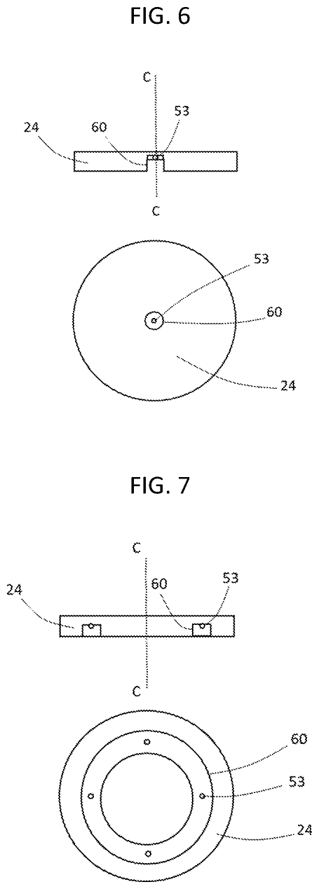

FIG. 6 illustrates a schematic side and bottom view of a simplified version of a lid unit 24 comprises one light 53 arranged in a light housing 60. The light may be a built in LED light. The one or more lights 53 may be positioned in the lid unit 24 within a radial distance from the central vertical axis C. FIG. 7 illustrates a schematic side and bottom view of a simplified version of a lid unit 24 comprising four built in LED lights distributed in an annular pattern in a light housing. The lights 53 are arranged evenly in a light housing 60 having an annular ring-shaped arranged in a distance from the central vertical axis C. Generally, the light housing may be arranged within a radial distance of 10-60 mm from the central vertical axis C.

The portable base 20 can for example be made from a polymeric material suitable for the operating temperatures of the irrigation liquid and/or for temperatures high enough to kill microorganisms.

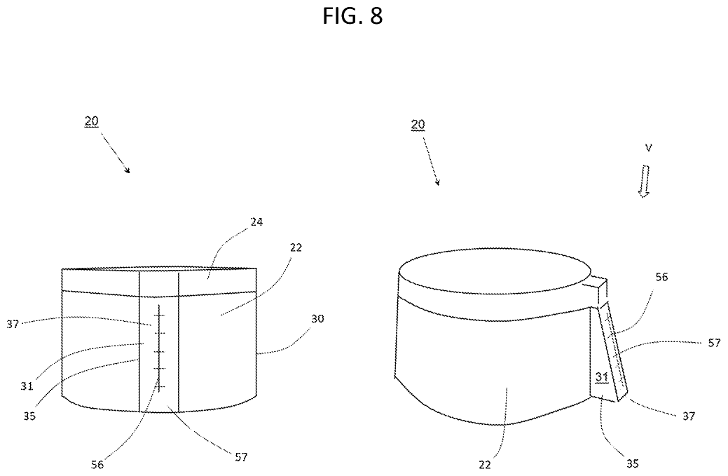

FIG. 8 illustrates a schematic side and a perspective view of a canister unit comprising an inclined protrusion 31. The inclination of the protrusion 31 in axial direction varies from the gradient of the side wall 30 of the canister unit 20.

The canister unit comprises a substantially cylinder-shaped side wall and, in the perspective view, the inclined protrusion 31 comprises a wedge-shape, where the inclined protrusion face 37 is adapted such that the distance in radial direction to the central vertical axis C increases towards the bottom portion 25 of canister unit 22. Thus, the protrusion face 37 will be visual and allow a user to view the protrusion face from a position above the portable base 20.
In axial direction, the protrusion face of the inclined protrusion is angled 2-45 degrees relative to the central vertical axis, such as 5-40, 6-35, 7-30, 8-25, 9-20, 10-15 degrees.
The protrusion face is connected to the side wall 30 by two vertical protrusion side faces 35.
The at least partly see-through portion 57 is arranged on the protrusion face 37 as well as the volumetric scale 56.
An arrow marked V illustrates the direction from where a user may view the portable base 20 during use, e.g., when the portable base is positioned on the floor next to a user sitting on a toilet.

Although particular features have been shown and described, it will be understood that they are not intended to limit the claimed invention, and it will be made obvious to those skilled in the art that various changes and modifications may be made without departing from the scope of the claimed invention. The specification and drawings are, accordingly, to be regarded in an illustrative rather than restrictive sense. The claimed invention is intended to cover all alternatives, modifications, and equivalents.

1. A portable base for a system for irrigating bowels of a user, where the portable base comprises:
a canister unit and a lid unit, the canister unit comprises a bottom portion, a side wall and a neck portion for forming an inner volume for containing an irrigation liquid;
wherein the neck portion circumscribes an annular opening around a central vertical axis of the portable base, the lid unit is adapted for closing of the annular opening of the canister unit, and the side wall comprises an at least partly see-through portion,
wherein the lid unit comprises one or more lights being adapted to illuminate the inner volume of the canister unit when the lid unit is secured to the canister unit.

2. The portable base according to claim 1, wherein the side wall comprises a radially extending inclined protrusion comprising a protrusion face, and the at least partly see-through portion is arranged on the protrusion face.

3. The portable base according to claim 1, wherein the canister unit comprises a volumetric scale arranged at the at least partly see-through portion of the side wall.

4. The portable base according to claim 1, wherein an entirety of the canister unit is made of a transparent see-through material.

5. The portable base according to claim 2, wherein the protrusion face of the inclined protrusion in axial direction is angled in a range between 10-15 degrees.

6. The portable base according to claim 2, wherein the protrusion face of the inclined protrusion is angled such that a radial distance measured from the central vertical axis C to the side wall is increasing towards the bottom portion of the canister unit.

7. The portable base according to claim 1, wherein the canister unit comprises a frustoconical shape.

8. The portable base according to claim 1, wherein at least ⅔ of the side wall of the canister unit in an axial direction is angled in a range between 10-15 degrees.

9. The portable base according to claim 1, wherein the neck portion is radially closer to the central vertical axis than the sidewall of the canister unit.

10. The portable base according to claim 1, wherein the one or more lights comprises LED Lights integrated in the lid unit.

11. The portable base according to claim 1, wherein one light is positioned in the lid unit within a radial distance of 30 mm or less from the central vertical axis.

12. The portable base according to claim 1, wherein the one or more lights are arranged evenly in an annular ring-shaped pattern in the lid unit within a radial distance of 10-60 mm from the central vertical axis.

13. The portable base according to claim 1, wherein the one or more lights emit a coloured light, with the coloured light selected from the group consisting of red light, blue light, green light, violet light, orange light, yellow light and white light.
